(19) United States
(12) Patent Application Publication
(10) Pub. No.: US 2017/0072027 A1
(43) Pub. Date: Mar. 16, 2017

(54) The Application of RESCAP to Attenuate and Prevent the Progression of Neurodegenerative Brain and Neuronal Diseases

(71) Applicant: AMRIF BV, Wageningen (NL)

(72) Inventors: Rudi Brands, Bunnik (NL); Willem Seinen, Bilthoven (NL)

(21) Appl. No.: 15/306,221

(22) PCT Filed: Apr. 30, 2015
(86) PCT No.: PCT/EP2015/059505

§ 371 Date: Oct. 24, 2016

(30) Foreign Application Priority Data
Apr. 30, 2014 (EP) 14166524.0

(51) Int. Cl. A61K 38/46

(52) U.S. Cl. CPC A61K 38/465; C12Y 301/03001

(57) ABSTRACT

The present invention relates to the treatment of neurodegenerative diseases, i.e. a group of chronic, progressive disorders characterized by the gradual loss of neurons in discrete areas of the central nervous system (CNS). Specifically, the present invention relates to alkaline phosphatase for use in the treatment of neurodegenerative disorders, preferably neurodegenerative disorders selected from the group consisting of Alzheimer's Disease; Parkinson's Disease; Amylotrophic Lateral Sclerosis; Multiple Sclerosis and Stroke.

The present invention relates to the treatment of neurodegenerative diseases, i.e. a group of chronic, progressive disorders characterized by the gradual loss of neurons in discrete areas of the central nervous system (CNS). Specifically, the present invention relates to alkaline phosphatase for use in the treatment of neurodegenerative disorders, preferably neurodegenerative disorders selected from the group consisting of Alzheimer's Disease; Parkinson's Disease; Amylotrophic Lateral Sclerosis; Multiple Sclerosis and Stroke.

Neurodegenerative diseases are a group of chronic, progressive disorders characterized by the gradual loss of neurons in discrete areas of the central nervous system (CNS). The mechanism (s) underlying their progressive nature remains unknown but substantial evidence has documented a common inflammatory mechanism in various neurodegenerative diseases (Gao and Hong 2008; Glass, Saijo et al. 2010).

Oral or parenteral supplementation of RESCAP supports the anti-inflammatory and neuro-protective function of alkaline phosphatase in the brain. Patients suffering from neurobehavioral diseases, like e.g. depression, myalgic encephalitis and schizophrenia, are also proposed to benefit from the anti-inflammatory and neuro-protective effects of RESCAP

The active principle of RESCAP is its alkaline phosphatase activity. In various animal models alkaline phosphatase has shown to attenuate the inflammatory response (Bentala, Verweij et al. 2002; Koyama, Matsunaga et al. 2002; Beumer, Wulferink et al. 2003; van Veen, van Vliet et al. 2005; van Veen, Dinant et al. 2006; Bates, Akerlund et al. 2007; Heemskerk, Masereeuw et al. 2009) (Bentala, Verweij et al. 2002; Koyama, Matsunaga et al. 2002; Beumer, Wulferink et al. 2003; Verweij, Bentala et al. 2004; van Veen, van Vliet et al. 2005; Su, Brands et al. 2006; Bates, Akerlund et al. 2007; Goldberg, Austen et al. 2008; Tuin, Poelstra et al. 2009; Bol-Schoenmakers, Fiechter et al. 2010; Whitehouse, Riggle et al. 2010).

Another function of alkaline phosphatase is protection of both epithelial and endothelial barriers in the body (Buchet 2013). In mucosal barrier systems but also in the blood brain barrier (BBB) the alkaline phosphatase activity is highly expressed. In the gastrointestinal tract alkaline phosphatase is reported to be involved in the maintenance of structure (Shao, Engle et al. 2000) and in protecting and restoring the gut barrier function (De Lisle, Mueller et al. 2011; Rentea, Liedel et al. 2012). We propose a comparable function of alkaline phosphatase in the BBB. Therefore we claim that in addition to the attenuation of neuro-inflammation in the brain, due to its intrinsic enzymatic activity, RESCAP will protect and restore the BBB function and protects the brain against neuro-inflammation triggered by the influx of pro-inflammatory factors from the systemic blood circulation.

In clinical studies it has been shown that alkaline phosphatase treatment improves renal function in severe sepsis or septic shock patients (Heemskerk, Masereeuw et al. 2009) and improves the disease activity scores in patients with moderate to severe ulcerative colitis (Lukas, Drastich et al. 2010)and in patients suffering from rheumatoid arthritis (Hammond 2014). In human volunteers exposed to 4 ng LPS/kg body weight (Beumer, Wulferink et al. 2003) and in patients undergoing cardiac artery bypass surgery (Kats, Brands et al. 2009), treatment with bovine RESCAP (bIAP) inhibited the induction of the pro-inflammatory cytokines TNFα, IFNy, IL6, and IL8, so preventing the initiation of a SIRS (systemic immune response syndrome) reaction and subsequent aggravation of clinical condition.

The systemic anti-inflammatory activity of alkaline phosphatase is now well established and therefore we claim that also in the central nervous system RESCAP will display its anti-inflammatory activity, reducing the inflammation in the CNS (neuroinflammation). Neuroinflammation is also a prominent feature shared by various neurodegenerative diseases, that drives the chronic progression of these diseases (Gao and Hong 2008; Glass, Saijo et al. 2010). Therewith RESCAP attenuates and prevents progression of neurodegenerative brain diseases.

Neurodegenerative diseases are characterized by slow progressive loss of neurons in the central nervous system (CNS), which leads to deficits in specific brain functions (e.g. memory, movement, cognition) performed by the affected CNS region. These neurodegenerative diseases include among others, Alzheimer's disease (AD), Parkinson's disease (PD), amyotrophic lateral sclerosis (ALS), multiple sclerosis (MS), Huntington's disease and multiple system atrophy. Neurodegenerative diseases usually extend over a decade and the actual onset of neurodegeneration may precede clinical manifestations by many years (McGeer and McGeer 2004).

A sustained inflammatory reaction is present in acute (e.g. stroke) and chronic (e.g. AD, PD, MS) neurodegenerative disorders (Marchetti and Abbracchio 2005). Each of these disorders is distinguished by a disease-specific mechanism for induction of inflammatory responses. The distinct pathways for the induction of inflammation and the specific anatomical locations at which these processes occur are likely determinants of the specific pathological features of each neurodegenerative disease. Remarkably, however, once induced there appears to be considerable convergence in the mechanisms that lead to amplification of inflammatory responses, neurotoxicity, and neuronal death (Glass, Saijo et al. 2010). Activation of innate immune cells in the CNS, such as microglia and astrocytes, is one of the universal components of neuroinflammation. In the diseased CNS, interactions between damaged neurons and dysregulated, overactivated microglia create a vicious self-propagating cycle causing uncontrolled, prolonged inflammation that drives the chronic progression of neurodegenerative diseases. (Marchetti and Abbracchio 2005) Microglia, the principal immune cells of the brain, is sensitive to a wide range of stimuli that can be released from damaged cells as a result of trauma, ischemia, toxic insults or, in general, changes in the physiological homeostasis (Hanisch and Kettenmann 2007). A major factor are nucleotides, amongst which ATP.

Extracellular ATP regulates the microglial branch dynamics in the intact brain, and its release from damaged tissue and surrounding astrocytes mediates a rapid microglial response towards brain injury. (Davalos, Grutzendler et al. 2005) ATP can reach high levels in the extracellular space as a consequence of release from both dying or abnormally functioning cells. It acts as a neuron-to-microglia alarm signal, through cell surface purinergic (P2) receptors widely distributed throughout the CNS. (D'Ambrosi, Finocchi et al. 2009)

In the postmortem brain of AD patients and various neurodegenerative disease animal models the expression and function of the P2X7 receptor (P2X7R), an ATP-gated ion channel abundantly expressed in microglia in the brain, is significantly up-regulated. (Yiangou, Facer et al. 2006; Matute, Torre et al. 2007; Ryu and McLarnon 2008; Diaz-Hernandez, Diez-Zaera et al. 2009; Takenouchi, Sekiyama et al. 2010; Volonte, Apolloni et al. 2012; Weisman, Camden et al. 2012) Blocking P2X7R using brilliant blue G, a P2X7R antagonist that can cross the blood-brain barrier, has been shown to result in the amelioration of neuropathology in various animal models. This supports the role of the P2X7R pathway in the progression of neurodegeneration. (Takenouchi, Sekiyama et al. 2010)

The physiological agonist of the P2X7R is ATP (Franke, Krugel et al. 2006; Takenouchi, Sekiyama et al. 2010). The ATP concentration in the extracellular space is in the low nanomolar range, but at sites of inflammation, tissue traumas, or intensive cell stimulation, its level can reach the low or even high micromolar range, whereas it is understood that actual ATP levels in the vicinity of the plasma membrane or at sites of close cell-to-cell contact can be much higher (Pellegatti, Falzoni et al. 2005; Pellegatti, Raffaghello et al. 2008) This suggests that ATP concentrations sufficient to activate even the low affinity P2X7 receptor may build up in vivo. Due to its proinflammatory activity, ATP is increasingly considered an early inflammatory mediator, or a “danger signal”(Di Virgilio 2005; Di Virgilio 2007), alerting the nervous tissue to aversive influences. It has an important role in inducing microgliosis and the control of microglial proliferation, migration and secretion of inflammatory mediators. It induces astrogliosis, with stellation, migration and proliferation of astrocytes and the release of molecules supporting tissue remodeling. (Abbracchio, Burnstock et al. 2009).

It is proposed that modulation of the P2X7R signaling pathway by an active reduction of the extracellular ATP concentration could be a therapeutic target for treating various neurodegenerative diseases. We claim that RESCAP will metabolize extracellular ATP into adenosine, a distress-relieving moiety engaged in anti-inflammatory activity (Eltzschig 2009; Eltzschig, Sitkovsky et al. 2012). The active principle of RESCAP is its alkaline phosphatase activity. Alkaline phosphatase can catalyze the entire hydrolysis chain from the nucleoside-5′-triphosphate to the respective nucleoside, it may scavenge the ligands of P2 receptors (e.g. P2X7R) and finally produce adenosine as the ligand of P1 receptors. (Zimmermann 2006; Zimmermann 2006).

In many inflammatory conditions it is shown that RESCAP safely and effectively target inflammatory mechanisms, also those that contribute to the pathogenesis of various neurodegenerative disorders. As neurodegenerative disorders, with the exception of stroke, are chronic diseases, it is likely that their prevention and treatment will require long-term therapy, imposing a corresponding requirement for a high level of safety. In clinical studies performed with alkaline phosphatase, the active ingredient of RESCAP, no signs of adverse activity have been observed in patients. Also in repeated dose toxicity studies with various animal species, that are immune-tolerant for this protein, the animals tolerated high dose daily intravenous injections with RESCAP. Therefore we expect that RESCAP can be safely applied in patients with neurodegenerative disorders.

However, to be clinically effective, anti-inflammatory therapeutics will have to gain access to the CNS in humans. As RESCAP is a high molecular weight protein, it will not easily pass the undisturbed blood brain barrier (BBB). Therefore a specific vehicle will be used to overcome the BBB in those diseases where the BBB is still functionally intact. However in several neurodegenerative disorders the permeability of the BBB is disturbed, which may allow RESCAP to cross this barrier to enter the brain directly from the blood circulation. Another way to target the specific cells and pathways important in the disease pathogenesis of neurodegenerative disorders is direct intrathecal administration of RESCAP.

### Examples of Neurodegenerative Diseases Targeted by RESCAP

### Alzheimer's Disease (AD)

AD is one of the most common age-related neurodegenerative diseases, with approximately 7% of people older than 65 years and about 40% of people older than 80 years being affected in industrialized countries. The symptoms of AD are characterized by loss of memory, progressive impairment of cognition, and various behavioral and neuropsychiatric disturbances. The pathological hallmarks of AD in the brain include extracellular amyloid plaques comprising aggregated, cleaved products of the amyloid precursor protein (APP) and intracellular neurofibrillary tangles (NETs) generated by hyperphosphorylated forms of the microtubule-binding protein tau. Evidence of an inflammatory response in AD includes changes in microglia morphologyfrom ramified (resting) to amoeboid (active)and astrogliosis (manifested by an increase in the number, size, and motility of astrocytes) surrounding the senile plaques. RESCAP will inhibit this ongoing inflammatory response and the progression of the neurodegeneration.

### Parkinson's Disease (PD)

Parkinson's disease (Pt)) is the second most common neurodegenerative disease after AD and is the most common movement disorder. Currently, about 2% of the population over the age of 60 is affected. Prominent clinical features are motor symptoms (bradykinesia, tremor, rigidity, and postural instability) and non-motor-related symptoms (olfactory deficits, autonomic dysfunction, depression, cognitive deficits, and sleep disorders). Like AD, PD is a proteinopathy; it is characterized by the accumulation and aggregation of misfolded α-synuclein. Neuropathological hallmarks are intracellular inclusions containing α-synuclein called Lewy bodies and Lewy neurites and the loss of dopaminergic neurons in the substantia nigra of the midbrain and in other brain regions as well (Braak, Del Tredici et al. 2003). Loss of dopaminergic neurons is not the only neuropathological alteration in PD, as microglial activation and an increase in astroglia and lymphocyte infiltration also occur. An increase in astroglial cells in post-mortem tissue from the brains of Pt) patients and an increased number of dystrophic astrocytes have also been reported (Braak, Sastre et al. 2007). Several lines of evidence suggest that inflammatory mediators derived from non-neuronal cells including microglia modulate the progression of neuronal cell death in PD (Hirsch and Hunot 2009). RESCAP will diminish the inflammation and progression of neuronal cell death.

### Amylotrophic Lateral Sclerosis

Amyotrophic lateral sclerosis (ALS), or Lou Gehrig's disease, is a progressive fatal neurodegenerative disease that affects motor neurons in the brainstem, spinal cord, and motor cortex. Clinical features involve degeneration of motor neurons producing fasciculation, muscle wasting and weakness, increased spasticity, and hyper-reflexia. Respiratory complications usually develop in patients with advanced disease, and the cause of death is generally paralysis of the respiratory muscles and diaphragm. With a projected lifetime risk of 1/2000, ALS is considered one of the most common motor neuron diseases (Eisen 2009). ALS is universally fatal, with a median age of onset of 55 years and a survival of 2-5 years after the onset of symptoms. Although the exact pathophysiological mechanisms underlying neurodegeneration in ALS remain uncertain, a common pathological hallmark is the presence of ubiquitin-immunoreactive cytoplasmic inclusions in degenerating neurons, followed by a strong inflammatory reaction (McGeer and McGeer 2002). Prominent neuroinflammation can be readily observed in pathologically affected areas of the CNS and in spinal cords from both human ALS patients and mouse models of the disease (McGeer and McGeer 2002). Typically, inflammation in ALS is characterized by gliosis and the accumulation of large numbers of activated microglia and astrocytes. The initial inflammatory reaction can come from extracellular ATP released by injured neurons, which is sensed by purinergic P2X7R on glia (Yiangou, Facer et al. 2006). RESCAP will dephosphorylate ATP, preventing purinergic signaling and progression of motor neuron degeneration.

### Multiple Sclerosis

Multiple sclerosis (MS) is an autoimmune disease that is characterized by inflammation, demyelination, and axon degeneration in the CNS. The clinical manifestations of MS include defects in sensation and in the motor, autonomic, visual, and cognitive systems. MS predominantly affects young adults and 2-3 times more females than males.

In the early stage of the disease, approximately 85% of MS patients show the relapse4emission type of disease. However, with time, the recovery of these relapsing-remitting patients is impaired and eventually leads to irreversible progression, that is, secondary progressive MS. The majority of relapsing-remitting MS patients progress to secondary progressive MS. In contrast, about 15% of MS patients do not show any remission, and the primary neurological symptoms exhibit continuous so-called primary progression (Mayo, Quintana et al. 2012) Both the adaptive and the innate immune system have been suggested to contribute to the pathogenesis and recovery from MS. Demyelination results from a primary defect in the immune system that targets components of the myelin sheath, resulting in secondary effects on neurons. MS lesions are characterized by infiltration of lymphocytes and antibody-producing plasma cells into the perivascular region of the brain and spinal cord white matter, an increase in microglia and astrocytes, and demyelination (Frischer, Bramow et al. 2009). When damage and the ensuing inflammatory response are transient, remyelination of nerves can take place as part of normal repair. However, in the presence of chronic inflammation, such as in MS, remyelination is severely impaired, and leads to axon degeneration and the eventual demise of the neuron (Glass, Saijo et al. 2010).

As progressive MS is invariably associated with inflammation ((Hanisch and Kettenmann 2007; Frischer, Bramow et al. 2009) and an emerging role of extracellular nucleotides is linked to the etiology of MS (Cieslak, Kukulski et al. 2011), we claim that RESCAP will inhibit the progression of MS. In mice immunized to induce experimental autoimmune encephalomyelitis (EAE) and treated with RESCAP for 7 days during different phases of disease, it was demonstrated that pre-symptomatic RESCAP treatment reduces neurological signs of EAE (Huizinga, Kreft et al. 2012).

### Stroke

A stroke, also known as cerebrovascular accident (CVA) is defined as the rapid loss of brain function due to a lack of blood supply. Stroke is the world's second leading cause of mortality, with a high incidence of severe morbidity in surviving victims. (Woodruff, Thundyil et al. 2011) A stroke may be ischemic (a blood vessel blocked by thrombosis or arterial embolism) or hemorrhagic (a leaking blood vessel). Ischemic strokes account for approximately 80% of all strokes, and are mainly caused by a blood clot that blocks blood flow to the brain. Thrombosis, embolism, and systemic hypoperfusion can decrease the blood supply, depriving neural cells of the glucose and oxygen they need to function. The bleeding that causes hemorrhagic stroke suddenly interferes with brain function. This bleeding can occur either within the brain or between the brain and the skull. Hemorrhagic strokes account for about 20% of all strokes, and are categorized depending on the site and cause of bleeding.

The damage by stroke is mainly caused by ischemia and reperfusion (I/R) resulting in irrecoverable, widespread neuronal death. Immediately after I/R astrocytes release ATP, inducing rapid activation of microglia that forms a barrier between the healthy and injured tissue (Davalos, Grutzendler et al. 2005). The release of ATP (and other nucleotides) is a key factor in the induction of inflammation, present hours to days after the initial insult and exacerbates the primary injury causing further brain damage. Thus, therapeutics which interfere with inflammatory processes in the CNS may have significant benefit for minimizing tissue damage and promoting neuronal survival after the primary insult. Recently a number of patents have been applied on P2X7 receptor antagonists for their potential to reduce central nervous system inflammation. (Friedle, Curet et al. 2010) We claim that RESCAP inhibits I/R damage and the subsequent inflammatory response in stroke by dephosphorylation of nucleotides and prevention of purinergic receptor signaling.

### Examples of Neuronal Diseases Targeted by RESCAP

### Myalgic Encephalomyelitis

Chronic fatigue syndrome (CFS), also known as myalgic encephalomyelitis (ME), has been widely studied over the past 25 years. Myalgia means muscle pain and encephalomyelitis means inflammation of the brain and spinal cord. Numerous mechanisms and theories have been proposed to explain its pathophysiology, epidemiology, clinical features and causation (Duivenboden, 2013). However, no single aetiology has been confirmed to fully explain the syndrome. Treatment for CFS focuses on symptom relief (Anderson, Jason, Hlavaty, Porter, & Cudia, 2012).

CFS or ME is classified as a nervous system disease by the World Health Organisation (WHO) since 1992 as multiple autoimmunity sources have been found to be linked with the disease. Increased levels have been found for both cytokines, like tumour necrosis factor a (TNF-α), interleukin-1 (IL-1) and nuclear factor-κβ(NF-κβ). Due to these adjusted levels dysregulation of inflammatory cytokines is a characteristic of ME/CFS (Morris, Berk, Galecki, & Maes, 2013). We claim that the anti-inflammatory effect as well as the protection of membrane barrier integrity by RESCAP improves the physical and mental condition in CFS.

### Other Neuronal Disorders

For more than a decade, inflammation has been implicated in chronic psychiatric disorders. Much of the key evidence demonstrating that inflammation and inflammatory mediators contribute to acute, chronic and psychiatric CNS disorders is summarised by Lucas et al. (2006) (Lucas, Rothwell et al. 2006). Acute phase proteins and cytokines such as IL-1β and IL-6 are elevated in the serum of depressed patients, and IL-lra and IFNy are increased in bipolar disorder. The aetiology of schizophrenia remains unexplained, but recently a vascular-inflammatory-genetic theory has been proposed (Hanson and Gottesman 2005), bringing together environmental and genetic factors that influence the inflammatory response and potentially contribute to the disease. Serum levels of many cytokines are increased in schizophrenia, including IL-1β and IL-6. We propose that RESCAP in these psychiatric disorders improves the clinical condition by inhibiting the local and systemic inflammation and preventing the influx of inflammatory triggers into the brain by maintaining the BBB integrity.

1. Alkaline phosphatase for use in the treatment of neurodegenerative disorders, preferably neurodegenerative disorders selected from the group consisting of Alzheimer's Disease; Parkinson's Disease; A mylotrophic Lateral Sclerosis; Multiple Sclerosis and Stroke.

2. Alkaline phosphatase for use in the treatment of neuronal and/or psychiatric disorders, preferably disorders selected from the group consisting of chronic fatigue syndrome, depression and schizophrenia.

3. Alkaline phosphatase for se according to claims 1, wherein said treatment comprises prevention of progression of said neurodegenerative and neuronal and/or psychiatric disease.
